SEQUENCE LISTING (2 sequences)

SEQ ID NO: 1 - DNA, 2507 bp, Clostridium difficile
gatcctatagaatttaacttagtaactggatggcaaactatcaatggtaaaaaatattattttgatataaatactggagcagctttaactagttataaaattattaatggtaaacacttttattttaataatgatggtgtgatgcagttgggagtatttaaaggacctgatggatttgaatattttgcacctgccaatactcaaaataataacatagaaggtcaggctatagtttatcaaagtaaattcttaactttgaatggcaaaaaatattattttgataataactcaaaagcagtcactggatggagaattattaacaatgagaaatattactttaatcctaataatgctattgctgcagtcggattgcaagtaattgacaataataagtattatttcaatcctgacactgctatcatctcaaaaggttggcagactgttaatggtagtagatactactttgatactgataccgctattgcctttaatggttataaaactattgatggtaaacacttttattttgatagtgattgtgtagtgaaaataggtgtgtttagtacctctaatggatttgaatattttgcacctgctaatacttataataataacatagaaggtcaggctatagtttatcaaagtaaattcttaactttgaatggtaaaaaatattactttgataataactcaaaagcagttaccggatggcaaactattgatagtaaaaaatattactttaatactaacactgctgaagcagctactggatggcaaactattgatggtaaaaaatattactttaatactaacactgctgaagcagctactggatggcaaactattgatggtaaaaaatattactttaatactaacactgctatagcttcaactggttatacaattattaatggtaaacatttttattttaatactgatggtattatgcagataggagtgtttaaaggacctaatggatttgaatattttgcacctgctaatacggatgctaacaacatagaaggtcaagctatactttaccaaaatgaattcttaactttgaatggtaaaaaatattactttggtagtgactcaaaagcagttactggatggagaattattaacaataagaaatattactttaatcctaataatgctattgctgcaattcatctatgcactataaataatgacaagtattactttagttatgatggaattcttcaaaatggatatattactattgaaagaaataatttctattttgatgctaataatgaatctaaaatggtaacaggagtatttaaaggacctaatggatttgagtattttgcacctgctaatactcacaataataacatagaaggtcaggctatagtttaccagaacaaattcttaactttgaatggcaaaaaatattattttgataatgactcaaaagcagttactggatggcaaaccattgatggtaaaaaatattactttaatcttaacactgctgaagcagctactggatggcaaactattgatggtaaaaaatattactttaatcttaacactgctgaagcagctactggatggcaaactattgatggtaaaaaatattactttaatactaacactttcatagcctcaactggttatacaagtattaatggtaaacatttttattttaatactgatggtattatgcagataggagtgtttaaaggacctaatggatttgaatactttgcacctgctaatacggatgctaacaacatagaaggtcaagctatactttaccaaaataaattcttaactttgaatggtaaaaaatattactttggtagtgactcaaaagcagttaccggactgcgaactattgatggtaaaaaatattactttaatactaacactgctgttgcagttactggatggcaaactattaatggtaaaaaatactactttaatactaacacttctatagcttcaactggttatacaattattagtggtaaacatttttattttaatactgatggtattatgcagataggagtgtttaaaggacctgatggatttgaatactttgcacctgctaatacagatgctaacaatatagaaggtcaagctatacgttatcaaaatagattcctatatttacatgacaatatatattattttggtaataattcaaaagcggctactggttgggtaactattgatggtaatagatattacttcgagcctaatacagctatgggtgcgaatggttataaaactattgataataaaaatttttactttagaaatggtttacctcagataggagtgtttaaagggtctaatggatttgaatactttgcacctgctaatacggatgctaacaatatagaaggtcaagctatacgttatcaaaatagattcctacatttacttggaaaaatatattactttggtaataattcaaaagcagttactggatggcaaactattaatggtaaagtatattactttatgcctga

SEQ ID NO: 2 - protein, 866 aa, Clostridium difficile
DPIEFNLVTGWQTINGKKYYFDINTGAALTSYKIINGKHFYFNNDGVMQLGVFKGPDGFEYFAPANTQNNNIEGQAIVYQSKFLTLNGKKYYFDNNSKAVTGWRIINNEKYYFNPNNAIAAVGLQVIDNNKYYFNPDTAIISKGWQTVNGSRYYFDTDTAIAFNGYKTIDGKHFYFDSDCVVKIGVFSTSNGFEYFAPANTYNNNIEGQAIVYQSKFLTLNGKKYYFDNNSKAVTGWQTIDSKKYYFNTNTAEAATGWQTIDGKKYYFNTNTAEAATGWQTIDGKKYYFNTNTAIASTGYTIINGKHFYFNTDGIMQIGVFKGPNGFEYFAPANTDANNIEGQAILYQNEFLTLNGKKYYFGSDSKAVTGWRIINNKKYYFNPNNAIAAIHLCTINNDKYYFSYDGILQNGYITIERNNFYFDANNESKMVTGVFKGPNGFEYFAPANTHNNNIEGQAIVYQNKFLTLNGKKYYFDNDSKAVTGWQTIDGKKYYFNLNTAEAATGWQTIDGKKYYFNLNTAEAATGWQTIDGKKYYFNTNTFIASTGYTSINGKHFYFNTDGIMQIGVFKGPNGFEYFAPANTDANNIEGQAILYQNKFLTLNGKKYYFGSDSKAVTGLRTIDGKKYYFNTNTAVAVTGWQTINGKKYYFNTNTSIASTGYTIISGKHFYFNTDGIMQIGVFKGPDGFEYFAPANTDANNIEGQAIRYQNRFLYLHDNIYYFGNNSKAATGWVTIDGNRYYFEPNTAMGANGYKTIDNKNFYFRNGLPQIGVFKGSNGFEYFAPANTDANNIEQAIRYQNRFLHLLGKIYYFGNNSKAVTGGWQTINGKVYYFMPDTAMAAAGGLFEDGVIYFFGVDGVKAPGIYG

(19) United States
(12) Patent Application Publication
(10) Pub. No.: US 2005/0202042 A1
(43) Pub. Date: Sep. 15, 2005

(54) Recombinant toxin A and toxin B protein carrier for polysaccharide conjugate vaccines

(76) Inventors: Tracy D. Wilkins, Riner, VA (US); David M. Lyerly, Radford, VA (US); J. Scott Moncrief, Christiansburg, VA (US); Danka Pavliakova, Pittsford, NY (US); Rachel Schneerson, Bethesda, MD (US); John B. Robbins, Chevy Chase, MD (US)

Correspondence Address: MORRISON & FOERSTER LLP, 3811 VALLEY CENTRE DRIVE, SUITE 500, SAN DIEGO, CA, 92130-2332, US

(21) Appl. No.: 10/987,508

(22) Filed: Nov. 12, 2004

Related U.S. Application Data
(63) Continuation of application No. 09/545,772, filed on Apr. 10, 2000.
(60) Provisional application No. 60/128,686, filed on Apr. 9, 1999.
(60) Provisional application No. 60/186,201, filed on Mar. 1, 2000.

(51) Int. Cl. A61K 39/116; A61K 31/739

(52) U.S. Cl. 424/203.1; 514/54

(57) ABSTRACT

The present invention provides for immunogenic compositions and their methods of use as vaccines and their method of preparation. These immunogenic compositions comprise a recombinant protein of toxin A or toxin B of Clostridium difficile conjugated to a polysaccharide of a microbial pathogen. The immunogenic compositions may include only a truncated portion of toxin A or toxin B, particularly the repeating units (rARU or rBRU), that is associated with a microbial pathogen polysaccharide. Such compositions are effective in eliciting T-cell dependent and antibody responses. These compositions are therefore effective as vaccines for humans, particularly children, and animals in affording protection against one or more microbial pathogens.

### CROSS-REFERENCE TO RELATED APPLICATIONS

This application is a continuation of U.S. Ser. No. 09/545,772 filed 10 Apr. 2000, which claims priority to U.S. Provisional Ser. No. 60/128,686, filed 9 Apr. 1999, and U.S. Provisional Ser. No. 60/186,201 filed 1 Mar. 2000.

### STATEMENT OF RIGHTS TO INVENTIONS MADE UNDER FEDERALLY SPONSORED RESEARCH

The experimental work disclosed herein was supported in part under U.S. Department of Health and Human Services funding agreement number SBIR R43 AI42457.

### TECHNICAL FIELD OF THE INVENTION

The present invention relates to the field of medical immunology and further to pharmaceutical compositions, methods of making and methods of use of vaccines. More specifically this invention relates to a recombinant protein derived from a gene encoding Clostridium difficile toxin A, or closely related toxin B, as a carrier protein for enhancing the immunogenicity of a polysaccharide antigen.

### BACKGROUND OF THE INVENTION

The development of effective vaccines has resulted in major advances for the prevention of many infectious diseases. Smallpox, for example, has been eliminated and the mention of polio, which has almost been completely eliminated, does not bring to the minds of younger generations the picture of crippling paralysis as it did several decades ago. The incidence of diphtheria, tetanus, measles, and whooping cough in many industrialized countries has been reduced significantly. Despite these advances, infectious diseases still remain the major cause of morbidity and mortality to the majority of persons around the world.

It is important that medical research continues to develop vaccines that are effective, inexpensive to produce and administer, and that exhibit minimal adverse side effects. Vaccination against pathogens is our first line of defense and represents a beneficial and cost-effective means of combating many infectious diseases. Therefore, it is imperative that collaborations such as the present one continue to develop new approaches for vaccines as well as improve those that we currently use.

Clostridium difficile, a Gram-positive anaerobic spore-forming bacillus, has been shown to be the etiologic agent of several forms of bacterial induced diarrhea. As part of a complex flora of the human intestinal tract, C. dificile has been shown to emerge as one of the causes of enteric microbial induced diarrhea following antibiotic therapy, which weakens or destroys many of the normal competitive enteric flora. Strains of C. difficile have been observed to cause only 25% of antibiotic-associated diarrheas, but have been found to be the causative agent of almost all cases of pseudomembranous colitis (“PMC”), some cases of which have been fatal (Lyerly, D. M. and T. D. Wilkins, in Infections of the Gastrointestinal Tract, Chapter 58, pages 867-891, (Raven Press, Ltd, New York 1995)). Additionally, C. difficile is frequently identified as a causative agent of nosocomial infectious diarrheas, particularly in older or immuno-compromised patients (U.S. Pat. No. 4,863,852 (Wilkins et al.) (1989)).

A significant component of the pathogenic repertoire of C. difficile is found in the two enteric toxins A and B produced by most strains (U.S. Pat. No. 5,098,826 (Wilkins et al.) (1992)). Toxin A is primarily an enterotoxin with minimal cytotoxic activity. While toxin B is a potent cytotoxin, the extensive damage to the intestinal mucosa is attributable to the action of toxin A, however, there are reports that toxins A and B may act synergistically in the intestine.

The genetic sequences encoding both toxigenic proteins A and B, the largest known bacterial toxins, with molecular weights of 308,000 and 269,000, respectively, have been elucidated (Moncrief et al., Infect. Immun. 65:1105-1108 (1997); Barroso et al., Nucl. Acids Res. 18:4004 (1990); Dove et al. Infect. Immun. 58:480-488 (1990)). Because of the degree of similarity when conserved substitutions are considered, these toxins are thought to have arisen from gene duplication. The proteins share a number of similar structural features with one another. For example, both proteins possess a putative nucleotide binding site, a central hydrophobic region, four conserved cysteines and a long series of repeating units at their carboxyl ends. The repeating units of toxin A, particularly, are immunodominant and are responsible for binding to type 2 core carbohydrate antigens on the surface of the intestinal epithelium (Krivan et al., Infect. Immun. 53:573-581 (1986); Tucker, K. and T. D. Wilkins, Infect. Immun. 59:73-78 (1991)).

The toxins share a similar molecular mechanism of action involving the covalent modification of Rho proteins. Rho proteins are small molecular weight effector proteins that have a number of cellular functions including maintaining the organization of the cytoskeleton. The covalent modification of Rho proteins is due to glucosyltransferase activity of the toxins. A glucose moiety is added to Rho using UDP-glucose as a cosubstrate (Just et al. Nature 375:500-503 (1995); Just et al. J. Biol. Chem 270:13932-13939 (1995)). The glucosyltransferase activity has been localized to approximately the initial 25% of the amino acid sequence of each of these toxins (Hofmann et al. J. Biol. Chem. 272:11074-11078 (1997); Faust and Song, Biochem. Biophys. Res. Commun. 251:100-105 (1998)) leaving a large portion of the toxins, including the repeating units, that do not participate in the enzymatic activity responsible for cytotoxicity.

The immunogenicity of the surface polysaccharides of bacterial pathogens is improved when these antigens are bound covalently to a carrier protein (conjugate). Conjugate vaccines against Haemophillus influenzae type b have virtually eliminated the disease in developed countries that routinely vaccinate children (Robbins, J. B., and R. Schneerson, J. Infect. Dis. 161:821-832 (1990); Robbins et al., JAMA 276:1181-1185 (1996)). This approach to improving the immunogenicity of polysaccharide antigens is based on experiments defining the effect of attaching a hapten (small molecule) or an antigen that is poorly immunogenic by itself to a carrier protein (Avery et al., J. Exp. Med. 50:521-533 (1929); Goebel, W. F., J. Exp. Med. 69:353-364 (1939); Buchanan-Davidson et al., J. Immunol. 83:543-555 (1959); Fuchs, et al., J. Biol. Chem. 240:3558-3567 (1965)). Conjugates containing polysaccharides from a number of different encapsulated pathogenic microorganisms have been tested in animals and humans and shown to elicit polysaccharide antibodies (Chu et al., Infect. Immun. 59:4450-4458 (1991); Devi et al., Infect. Immun. 59:732-736 (1991); Devi et al., Infect. Immun. 59:3700-3707 (1990); Fattom et al., Infect. Immun. 60:584-589 (1992); Fattom et al., Infect. Immun. 61:1023-1-32 (1993); Konadu et al., Infect. Immun. 62:5048-5054 (1994); Kayhty et al. J. Infect. Dis. 172:1273-1278 (1995); Szu et al., Infect. Immun. 54:448-453 (1986); Szu et al., Infect. Immun. 59:4555-4561(1991); Szu et al., Infect. Immun. 57:3823-3827 (1989)). Antibodies to surface polysaccharides induced by vaccination with conjugates may confer protection against the encapsulated microorganism by inactivating the innoculum (Robbins et al. J. Infect. Dis. 171:1387-1398 (1995)).

Most carriers for conjugate vaccines have been medically useful proteins, namely, inactivated toxins of: tetanus, diphtheria, pertussis and Pseudomonas aeruginosa (Anderson et al. J. Clin. Invest. 76:52-59 (1985); Cohen et al. Lancet 349:155-159; Dagan et al. Infect. Immun. 66:2093-2098 (1998); Devi et al. Proc. Natl. Acad. Sci USA 88:7175-7179 (1991); Pavliakova et al. Infect. Immun. 67:5526-5529 (1999); Schneerson et al. Infect. Immun. 60:3528-3532 (1992)). Conjugate vaccines, therefore, may confer protection against pathogens whose protective antigens are the carrier proteins, including those that cause toxin-mediated diseases. In cases where tetanus toxin has been used, toxin-neutralizing antibody responses have been observed (Claesson et al. J. Pediatr. 112:695-702 (1988); Lagergard et al. Infect. Immun. 58:687-694 (1990); Schneerson et al. Infect Immun. 52:519-528 (1986)). Further, tetanus toxin (molecular weight 150,000) is twice the size of either diphtheria toxin or exotoxin A from Pseudomonas aeruginosa and results in a higher level of antibody produced against the polysaccharide antigenic component (Robbins, J. B. and R. Schneerson, J. Infect. Dis., 161:821-832 (1990)).

Proteins derived from toxin A and B of C. difficile may be candidates for a carrier protein that may be useful for conjugate vaccines against nosocomial infections by serving as effective carriers for polysaccharides. Examples of encapsulated nosocomial pathogens that could likely be protected against by rARU conjugate vaccines include: Staphylococcus aureus; coagulase-negative Staphylococcus; Enterococcus species; Enterobacter species; Candida species; group B Streptococcus; Escherichia coli; and Pseudomonas species.

Nosocomial infections due to S. aureus and C. difficile represent a major health care problem in the United States. This is particularly true in light of the emerging threat posed by antibiotic resistant pathogens such as methicillin resistant S. aureus (MRSA) and vancomycin resistant Enterococci (VRE) (Thornsberry C. West J. Med. 164:28-32 (1996) that may transfer resistance to MRSA. The incidence of S. aureus infections continues to rise and it is currently the most common cause of death from nosocomial infections (Weinstein, R A Eme1998). Its prevalence, in part, is due to the wide range of infections it causes and its extensive repertoire of virulence factors (Archer, G L Clin. Infect. Dis. 26:1179-1181 (1998)). Further, strains of S. aureus are commonly carried in the nasal passages and on the skin making it exceedingly difficult to control the spread of this organism. In addition to causing hospital-acquired infections, S. auerus is becoming more commonly recognized as a community-acquired infection (Kayaba et al. Surg Today 27:217-219 (1997); Moreno et al. Clin Infect. Dis. 212:1308-1312(1995)). Strains of S. aureus that are increasingly virulent and resistant to antibiotic therapy continue to emerge. Recently strains with intermediate resistance to vancomycin have been identified in the U.S. and other developed nations (Tenover et al. J. Hosp Infect 43 Suppl:S3-7 (1999); Woodford et al. J. Antimicrob Chemother. 45:258-259 (2000)). This is an alarming development, since vancomycin resistant strains of S. aureus that are also multiply resistant to other antibiotics would be exceedingly difficult to treat without the development of novel therapies.

Serotypes 5 and 8 cause about 85% of S. aureus infections and experimental evidence suggests that antibodies to capsular polysaccharides of S. aureus may protect against disease (Fattom et al. Infect. Immun. 58:2367-2374 (1990); Fattom et al. Infect. Immun. 61:1023-1024 (1993)). Therefore, a conjugate vaccine against serotypes 5 and 8 may be broadly protective. Further, in the case of H. influenzae type b (Hib) conjugate vaccines, vaccination has decreased the carriage of H. influenzae in the nasal passages. This is thought to have contributed to the success of Hib conjugate vaccines through herd immunity (Robbins et al. JAMA 276:1181-1185 (1996)). A similar effect may be seen with an effective conjugate vaccine against S. aureus, which may be particularly important for eliminating hospital acquired infections by vaccinating health care workers as well as patients.

Conjugate vaccines are also considered to provide epitopes to polysaccharide antigens that may be recognized by T helper cells (Avery O. T. and W. F. Goebel J. Experimental Med. 50:533-550 (1929)). A strong antibody response appears to require an interaction of antigen-specific B cells with T helper cells. This event is thought to be essential in a humoral immune response that leads to production of large amounts of high avidity antibodies and the formation of immunological memory. In this event B cells act as antigen presenting cells (APCs). Unlike other APCs, however, B cells take up antigen in a specific manner by binding the antigen with antibodies on the surface of the cell. These B cells are capable of differentiating into plasma cells that secrete antibody to the antigen. Also, a subpopulation of activated B cells differentiate into memory cells that are primed to recognize the antigen and become activated upon subsequent exposure. In both cases differentiation requires direct interaction with T helper cells. Upon uptake of the antigen, B cells process the antigen (protein) and present T cell epitopes on the surface in context with MHC class II. Antigen specific T helper cells then bind the T helper epitope/MHC class II complex and release helper cytokines leading to the differentiation of B cells into antibody secreting plasma cells or memory cells. The event also leads to differentiation of the specific T helper cells into memory cells. The immune system is therefore primed for an anamnestic response (booster effect) upon subsequent exposure to the antigen.

Polysaccharide antigens do not contain T cell epitopes. Polysaccharides, therefore, induce a T cell-independent response when presented without an attached protein. The T cell-independent response results in short lived antibody responses characterized by low affinity antibodies predominated by IgM. Conjugation of a protein to the polysaccharide provides T cell epitopes to the polysaccharide. This converts the T cell-independent response to a T cell-dependent response. Upon uptake of the conjugate by B cells specific for the polysaccharide the protein portion of the conjugate is processed and T cell epitopes are displayed on the surface of the B cell in context with MHC class II for interaction with T helper cells. Therefore, B cells that secrete antibody to the polysaccharide are expanded in a T cell-dependent manner.

rARU is comprised of 31 contiguous repeating units and may contain multiple T cell epitopes (Dove et al. Infect. Immun. 58:480-488 (1990). The repeating units are defined as class I and class II repeats. rARU may be uniquely suited for use in inducing T cell-dependent response to polysaccharides. The sequence of each unit is similar but not identical.

The toxin B repeating units have similar features to those of rARU. Like rARU, the recombinant toxin B repeating units (rBRU) are relatively large (˜70 kDa) and are composed of contiguous repeats of similar amino acid sequences (Barroso et al. Nucleic Acids Res. 18:4004 (1990); Eichel-Streiber et al. Gene 96:107-113 (1992)). Less is known about this portion of toxin B than the binding domain of toxin A. Thomas et al (U.S. Pat. No. 5,919,463 (1999)) disclose C. difficile toxin A or toxin B or certain fragments thereof as mucosal adjuvants intranasally administered to stimulate an immune response to an antigen (e.g., Helicobacter pylori urease, ovalbumin (OVA), or keyhole limpet hemocyanin (KLH)).

Even were one to consider rARU and rBRU as candidate carrier proteins for conjugate vaccines, the production of such proteins presents certain challenges. There are methods for the production of toxin A and antibodies elicited thereto (U.S. Pat. No. 4,530,833 (Wilkins et al.)(1985); U.S. Pat. No. 4,533,630 (Wilkins et al.)(1985); and U.S. Pat. No. 4,879,218 (Wilkins et al.)(1989)). There are significant difficulties in producing sufficient quantities of the C. difficile toxin A and toxin B proteins. These methods are generally cumbersome and expensive. However, the present invention provides for the construction and recombinant expression of a nontoxic truncated portions or fragments of C. difficile toxin A and toxin B in strains of E. coli. Such methods are more effective and commercially feasible for the production of sufficient quantities of an efficient carrier molecule for raising humoral immunogenicity to polysaccharide antigens.

Part of the difficulty that the present invention overcomes concerns the fact that large proteins are difficult to express at high levels in E. coli. Further, an unusually high content of AT in these clostridial gene sequences (i.e., AT-rich) makes them particularly difficult to express at high levels (Makoff et al. Bio/Technology 7:1043-1046 (1989)). It has been reported that expression difficulties are often encountered when large (i.e., greater than 100 kd) fragments are expressed in E. coli. A number of expression constructs containing smaller fragments of the toxin A gene have been constructed, to determine if small regions of the gene can be expressed to high levels without extensive protein degradation. In all cases, it was reported that higher levels of intact, full length fusion proteins were observed rather than the larger recombinant fragments (Kink et al., U.S. Pat. No. 5,736,139; see: Example 11(c)). It has been further reported that AT-rich clostridial genes contain rare codons that are thought to interfere with their high-level expression in E. coli (Makoff et al. Nucleic Acids Research 17:10191-10202). The present invention provides for methods to produce genes that are both large and AT-rich. For example, the toxin A repeating units are approximately 98 kDa and the gene sequence has an AT content of approximately 70% that is far above the approximately 50% AT content of the E. coli geneome. The present invention provides for methods of expressing AT-rich genes (including very large ones) at high levels in E. coli without changing the rare codons or supplying rare tRNA.

Citation of the above documents is not intended as an admission that any of the foregoing is pertinent prior art. All statements as to the date or representation as to the contents of these documents is based on the information available to the applicants and does not constitute any admission as to the correctness of the dates or contents of these documents. Further, all documents referred to throughout this application are incorporated in their entirety by reference herein. Specifically, the present application claims benefit of priority to U.S. provisional patent application Ser. No. 60/186,201, which was filed on Mar. 1, 2000, and U.S. provisional patent application Ser. No. 60/128,686, which was filed on Apr. 9, 1999, and which provisional patent applications are incorporated in their entirety by reference herein.

### SUMMARY OF THE INVENTION

The present invention is drawn to an immunogenic composition that includes a recombinant protein component and a polysaccharide component. The gene encoding the protein component is isolated from a strain of C. difficile. The polysaccharide component is not a C. difficile polysaccharide and is isolated from a source other than C. difficile.

A preferred embodiment of this invention provides that the protein component is a toxin or a toxin fragment. A further preferred embodiment provides that the toxin is C. difficile toxin A. A more preferred embodiment of the present invention provides that the protein component comprise all the amino acid sequence of the C. difficile toxin A repeating units (rARU) or fragment thereof. The immunogenic composition may further include a pharmaceutically acceptable carrier or other compositions in a formulation suitable for injection in a mammal.

Another preferred embodiment provides that the toxin is C. difficile toxin B. A further preferred embodiment provides that the protein is comprised of a portion of toxin B that includes the repeating units (rBRU) of the toxin or a fragment thereof.

Another embodiment of the present invention includes methods for producing an immunogenic composition by: constructing a genetic sequence encoding a recombinant protein component where the gene encoding the protein component is isolated from a strain of C. difficile; expressing the recombinant protein in a microbial host; recovering the recombinant protein component from a culture of the microbial host; conjugating the protein component to a polysaccharide component, where the polysaccharide component is isolated from a source other than C. difficile; and recovering the conjugated protein component and polysaccharide component. A preferred embodiment provides that the polysaccharide component is isolated from a pathogenic microorganism or is chemically synthesized. A still further preferred embodiment of this invention includes maintaining expression of the genetic sequence encoding the protein component in the microbial host throughout the growth of the host cell by constant and stable selective pressure.

A further preferred embodiment of this invention provides that the pathogenic microorganism is selected from the group consisting of: Streptococcus pneumoniae; Neisseria meningitidis; Escherichia coli; and Shigella species. An even further preferred embodiment is that the pathogenic microorganism consists of an encapsulated microbial pathogen that causes nosocomial infections including: Staphylococcus aureus; coagulase- negative Staphylococcus species; Enterococcus species; Enerobacter species; Candida species; Escherichia coli; and Pseudomonas species.

Another embodiment of this invention includes an expression vector and transformed microbial host cell, where the expression vector comprises the gene encoding the protein component. A preferred embodiment provides that the gene encoding the protein component is operably linked to one or more controllable genetic regulatory expression elements. An even further preferred embodiment provides that the gene encoding the protein component is fused to a second genetic sequence, the expression of which results in the production of a fusion protein. A still further preferred embodiment includes that the controllable genetic regulatory expression elements comprise an inducible promoter sequence that is operatively positioned upstream of the gene encoding the protein component and the inducible promoter sequence is functional in the microbial host. An even further preferred embodiment of the present invention includes a selective phenotype encoded on the expression vector by an expressible genetic sequence, the expression of which in the microbial host results in stable growth of the microbial host and constant production of the protein component when the host is cultured under conditions for which the selective phenotype is necessary for growth of the microbial host. A still further preferred embodiment includes a selectable phenotype that confers drug resistance upon the microbial host, while an even further preferred embodiment provides that the drug resistance gene is a kanamycin resistance gene, the expression of which enables the microbial host to survive in the presence of kanamycin in the culture medium.

The methods and compositions of the present invention also provide for a level of expression of the recombinant protein in the microbial host at a level greater than about 10 mg/liter of the culture, more preferably greater than about 50 mg/liter and even more preferably at 100 mg/liter or greater. The molecular weight of the protein is greater than about 30 kDa, preferably greater than about 50 kDa and even more preferably greater than about 90 kDa. This invention also provides that the protein may be recovered by any number of methods known to those in the art for the isolation and recovery of proteins, but preferably the recovery is by ammonium sulfate precipitation followed by ion exchange chromatography.

The present invention further includes methods for preparing the immunogenic composition that provides that the protein is conjugated to the polysaccharide by one of a number of means known to those in the art, but preferably by first derivatizing the protein by succinylation and then conjugating the polysaccharide component to the protein through a reaction of the protein and polysaccharide component with 1, ethyl-3-(3-dimethylaminopropyl) carboiimide hydrochloride. Additionally the invention contemplates the activation of the polysaccharide component by the use of any of several reagents, but preferably cyanogen bromide. The polysaccharide may be further derivatized by adipic acid dihydrazide. Conjugates synthesized with rARU may also be prepared by reductive amination or any other methods known in the art (Gray G R Methods Enzymol 50:155-160 (1978); Pawlowski et al. Vaccine 17:1474-1483).

The present invention further includes methods of use of compositions of this invention for the treatment of mammalian subjects infected with a pathogenic microorganism. Similarly, this invention provides methods of use of compositions of the present invention to provide protection against infection of a mammalian subject by a pathogenic microorganism.

### BRIEF DESCRIPTION OF THE DRAWINGS

FIG. 1 shows a schematic of Clostridium difficile toxins A and B. The enzymatic activity responsible for the cytotoxicity of toxins A and B is contained in the N-terminal glucosylyltransferase domain (Just et al. Nature 375:500-503 (1995); Just et al. J. Biol. Chem 270:13932-13939 (1995)). A DXD motif common to glycosyltransferases is essential for enzymatic activity (Busch et al. J. Biol. Chem 273:19566-19572 (1998)). The enzymatic domain and middle region of the toxin are deleted from the toxin A gene fragment encoding rARU (toxin A repeating units comprising the binding domain). The small open box at the end of toxin A represents a small stretch of hydrophobic amino acids.

FIG. 2 shows the nucleotide sequence (numbers 5690-8293, GenBank accession number M30307, Dove et al. 1993) of the toxin A gene region that encodes rARU and the toxin A stop codon. The sequence encodes for the entire repeating units of toxin A from C. difficile strain VPI 10463 as defined by Dove et al. (Dove et al., Infect Immun. 58:480-488 (1990)). In addition it encodes for 4 amino acids upstream of the beginning of the repeating units and a small stretch of hydrophobic amino acids at the end of toxin A. The Sau3A site (underlined) at the beginning of the sequence was used to subclone the gene fragment to an expression vector. The stop codon at the end of the sequence is italicized.

FIG. 3 shows the amino acid sequence (GenBank accession number M303307) of rARU. The invention contemplates the use of any recombinant protein containing this amino acid sequence, any fragment therein, any fusion protein containing rARU or a fragment therein, and any larger fragment from toxin A carrying all or part of rARU, as a carrier for conjugate vaccine compositions.

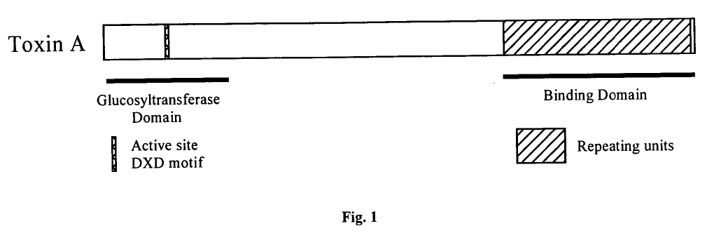

FIG. 4 shows the expression vector pRSETB-ARU-Km^{r} used for expression of rARU. A Sau3A/HindIII gene fragment of approximately 2.7 kb containing the entire nucleotide sequence encoding rARU, stop codon, and a small region downstream of the toxin A stop codon, was subcloned to the vector pRSETB digested with BamHI and HindIII. In a subsequent step the kanamycin resistance gene was subcloned at the HindIII site located downstream of the rARU gene fragment. The 1.2 kb fragment encoding the Km^{r} gene was derived from pUC4K (GenBank accession number X06404) by digestion with EcoRI and subcloned at the HindIII site after blunt ending of the vector and Km^{r} cassette with Klenow fragment. Expression vector pRSETB-ARU-Km^{r} was transformed into BL21(DE3) for expression of rARU under control of the T7 promoter.
HindIII/EcoRI sites were eliminated by blunt ending.

FIG. 5 shows an SDS-PAGE gel (15% acrylamide) of rARU expression and purification steps. Lanes: 1) 4 μl of 10× BL21(DE3) E. coli/pRSETB-ARU-Km^{r} lysate 2) 4 μl of dialyzed 40% ammonium sulfate fraction at 10× relative to the original culture volume 3) 5 μl rARU (0.88 mg/ml) purified by CL-6B Sepharose anion exchange chromatography.

FIG. 6 shows the chemical structure of polysaccharides conjugated to rARU. Pneumococcal type 14 is a neutral high molecular weight branched copolymer (Lindberg et al. Carbohydr. Res. 58:177-186 (1977)), Shigella flexneri 2a O-specific polysaccharide is a comparatively lower molecular weight neutral branched copolymer (Carlin et al. Eur. J. Biochem. 139:189-194 (1984); Kenne et al. Eur. J. Biochem. 91:279-284 (1978)), and each subunit of E. coli K1, a linear high molecular weight homopolymer, is negatively charged (Bhattacharjee et al. J. Biol. Chem. 250:1926-1932 (1975)). Conjugation of each polysaccharide to rARU resulted in high-level antibody responses. Thus, the use of rARU as a carrier is likely to be applicable to all polysaccharides.

### DETAILED DESCRIPTION OF THE INVENTION

The present invention is drawn to an immunogenic composition that includes a recombinant protein component and a polysaccharide component. The gene encoding the protein component is isolated from a strain of C. difficile. The polysaccharide component is not a C. difficile polysaccharide and is isolated from a source other than C. difficile. The polysaccharide is medically useful and is isolated from a pathogenic microorganism or synthesized. A preferred embodiment of this invention provides that the protein is a toxin or a toxin fragment. An even further preferred embodiment provides that the toxin is toxin A, with yet a further preferred embodiment being a portion of the toxin containing all of the amino acid sequence of the toxin A repeating units (rARU) or fragment thereof. Another preferred embodiment is that the toxin is toxin B, with yet another preferred embodiment being a portion of the toxin containing all of the amino acid sequence of the repeating units (rBRU) or a fragment thereof. The immunogenic composition may further include a pharmaceutically acceptable carrier or other compositions in a formulation suitable for injection in a mammal.

These immunogenic compositions of the present invention elicit an immune response in a mammalian host, including humans and other animals. The immune response may be either a cellular dependent response or an antibody dependent response or both and further the response may provide immunological memory or a booster effect or both in the mammalian host. These immunogenic compositions are useful as vaccines and may provide a protective response by the mammalian subject or host to infection by a pathogenic microorganism.

The present invention further includes methods for producing an immunogenic composition by: constructing a genetic sequence encoding a recombinant protein, where the gene encoding the protein is isolated from a strain of C. difficile; expressing the recombinant protein in a microbial host; recovering the recombinant protein from a culture of the host; conjugating the protein to a polysaccharide component, wherein the polysaccharide component is isolated from a source other than C. difficile; and recovering the conjugated protein and polysaccharide component. The protein component may also consist of a fusion protein, whereby a portion of the said recombinant protein is genetically fused to another protein. Preferably the expression of the genetic sequence is regulated by an inducible promoter that is operatively positioned upstream of the sequence and is functional in the host. Even further, the said genetic sequence is maintained throughout the growth of the host by constant and stable selective pressure. Maintenance of the expression vector may be conferred by incorporation in the expression vector of a genetic sequence that encodes a selective genotype, the expression of which in the microbial host cell results in a selective phenotype. Such selective genotypes, include a gene encoding resistance to antibiotics, such as kanamycin. The expression of this selective genotypic sequence on the expression vector in the presence of a selective agent or condition, such as the presence of kanamycin, results in stable maintenance of the vector throughout growth of the host. A selective genotype sequence could also include a gene complementing a conditional lethal mutation.

Other genetic sequences may be incorporated in the expression vector, such as other drug resistance genes or genes that complement lethal mutations.

Microbial hosts of this invention may include: Gram positive bacteria; Gram negative bacteria, preferably E. coli; yeasts; filamentous fungi; mammalian cells; insect cells; or plant cells.

The methods of the present invention also provide for a level of expression of the recombinant protein in the host at a level greater than about 10 mg/liter of the culture, more preferably greater than about 50 mg/liter and even more preferably at 100 mg/liter or greater than about 100 mg/liter. The molecular weight of the protein is greater than about 30 kDa, preferably greater than about 50 kDa and even more preferably greater than about 90 kDa. This invention also provides that the protein may be recovered by any number of methods known to those in the art for the isolation and recovery of proteins, but preferably the recovery is by ammonium sulfate precipitation followed by ion exchange chromatography.

The present invention further includes methods for preparing the immunogenic composition that provides that the protein is conjugated to the polysaccharide by one of a number of means known to those in the art, but preferably by first derivatizing the protein by succinylation and then conjugating the polysaccharide component to the protein through a reaction of the protein and polysaccharide component with 1, ethyl-3-(3-dimethylaminopropyl)carboiimide hydrochloride. Additionally the invention contemplates the activation of the polysaccharide component by the use of any of several reagents, but preferably cyanogen bromide. The polysaccharide may be further derivatized by adipic acid dihydrazide.

A number of polysaccharides components may be selected and conjugated to the protein component of the present invention. The immunogenic compositions of the present invention may further comprise a polysaccharide, lipopolysaccharide, capsular polysaccharide or other polysaccharide component. Such polysaccharide component may be selected, for example, from a pathogenic microorganism selected from the group consisting of: Streptococcus pneumoniae; Shigella species; and Escherichia coli.

Such polysaccharide components may be more specifically selected, for example, from a serotype of Streptococcus pneumoniae, selected from the group consisting of serotypes: 1, 2, 3, 4, 5, 6B, 7F, 8, 9N, 9V, 10A, 11A, 12F, 14, 15B, 17F, 18C, 19A, 19F, 20, 22F, 23F, 25, and 33F. Also, the polysaccharide component may be selected from any species of Shigella, including, for example, S. flexneri and may include any serotype of a Shigella species, including S. flexneri, serotype 2a. The polysaccharide may be specifically selected from a type of E. coli, for example E. coli K1.

The polysaccharide component may also be selected from any nosocomial pathogenic microorganism, from the group consisting of: Staphylococcus aureus; coagulase-negative Staphylococcus species; Enterococcus species; Enterobacter species; Candida species; group B Streptococcus; Escherichia coli; and Pseudomonas species.

Polysaccharide components may be more specifically selected, for example, from serotypes of S. aureus, including, for example, S. aureus serotype 5 or S. aureus serotype 8.

Also, high yields of recombinant protein may be dependent on the growth conditions, the rate of expression, and the length of time used to express the AT-rich gene. In general, AT-rich genes appear to be expressed at a higher level in E. coli during a post-exponential or slowed phase of growth. High-level production of the encoded protein requires moderate levels of expression over an extended period (e.g. 20-24 h) of post-exponential growth rather than the typical approach of high-level expression during exponential growth for shorter periods (e.g. 4-6 h). In this regard, it is more efficient to maintain plasmids carrying the gene of interest by maintaining constant selective pressure for the gene or its expression vector during the extended period of growth. One aspect of the present invention is using an antibiotic that is not inactivated or degraded during growth of the expression host cell as is found with ampicillin. This embodiment involves the expression of genes encoding resistance to kanamycin as the selective phenotype for maintaining the expression vector which comprises such kanamycin resistance genetic sequences. Expression of large AT-rich clostridial genes in E. coli at levels (>100 mg/liter) provided for by methods of the present invention was hitherto unknown.

Terms as used herein are based upon their art recognized meaning and should be clearly understood by the ordinary skilled artisan.

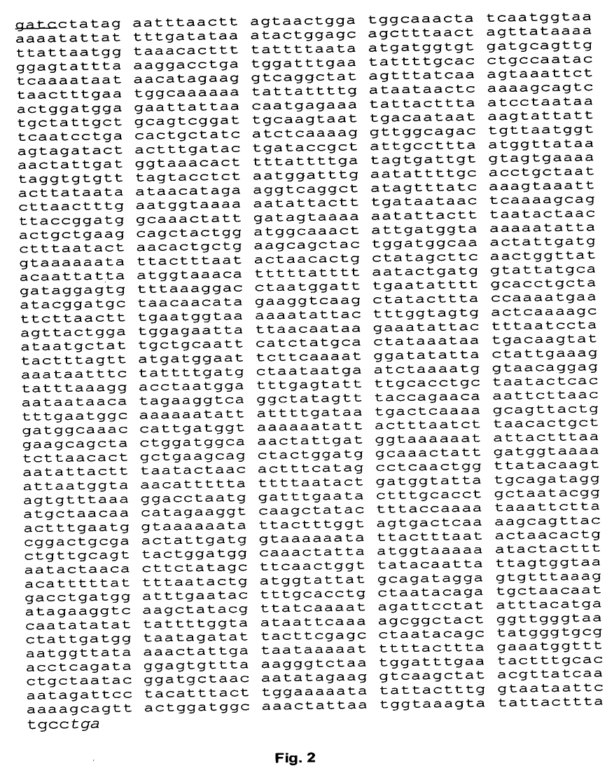

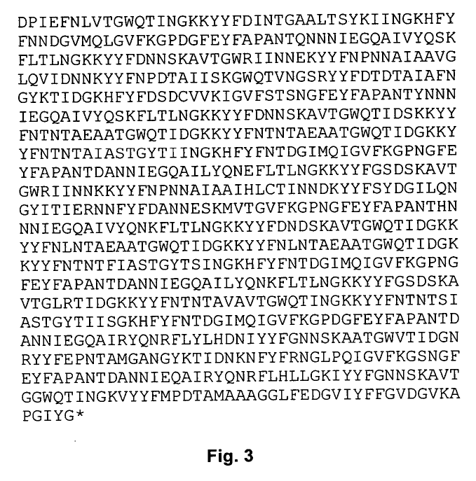

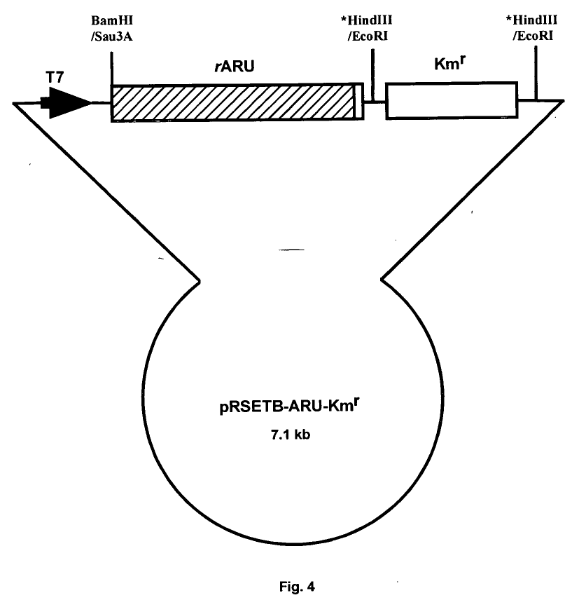

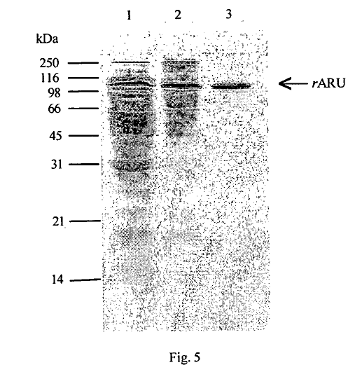

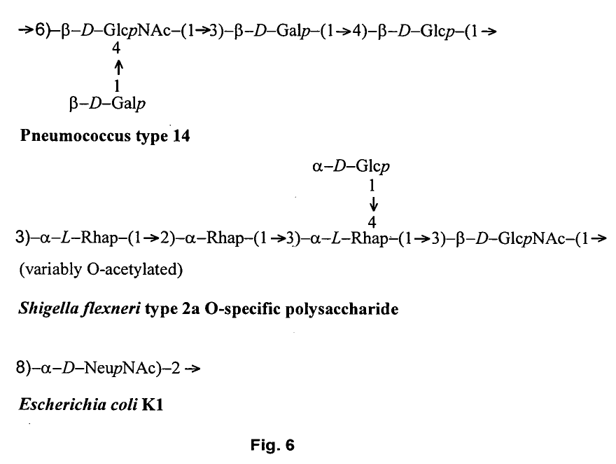

rARU is a recombinant protein containing the repeating units of Clostridium difficile toxin A as defined by Dove et al. (Dove et al. Infect. Immun. 58:480-488 (1990)). The nucleotide sequence encoding rARU and the amino acid sequence of rARU are shown in FIGS. 2 and 3, respectively. The rARU expressed by pRSETB-ARU-Km^{r} contains the entire repeating units region of toxin A. The invention further contemplates the use of this recombinant protein, or any other protein containing the entire repeating units of toxin A or any fragment therein, whether expressed alone or as a fusion protein.

A fusion protein is a recombinant protein encoded by a gene or fragment of a gene, genetically fused to another gene or fragment of a gene.

An immunogenic composition is any composition of material that elicits an immune response in a mammalian host when the immunogenic composition is injected or otherwise introduced. The immune response may be humoral, cellular, or both.

A booster effect refers to an increased immune response to an immunogenic composition upon subsequent exposure of the mammalian host to the same immunogenic composition.

A humoral response results in the production of antibodies by the mammalian host upon exposure to the immunogenic composition.

Having now generally described the invention, the same will be more readily understood through reference to the following examples which are provided by way of illustration, and are not intended to be limiting of the present invention, unless specified.

### EXAMPLES

### Example 1

Construction of rARU Expression Vector.

The vector pRSETB-ARU-Km^{r} used for expression and purification was constructed using standard techniques for cloning (Sambrook et al., Molecular Cloning: A Laboratory Manual (1989)). The nucleotide sequence of the toxin A gene fragment encoding rARU was derived from the cloned toxin A gene (Dove et al., Infect. Immun. 58:480-488 (1990); Phelps et al., Infect Immun. 59:150-153 (1991)) and is shown in FIG. 2. The gene fragment encodes a protein 867 amino acids in length (FIG. 3) with a calculated molecular weight of 98 kDa. The gene fragment was subcloned to the expression vector pRSETB. A kanamycin resistance gene was subsequently subcloned to the vector. The resulting vector pRSETB-ARU-Km^{r} expresses rARU. An additional 31 amino acids at the N-terminus of the recombinant protein are contributed by the expression vector pRSETB. The final calculated molecular weight of the recombinant protein is 102 kDa.

### Example 2

Expression and Purification of rARU.

Escherichia coli T7 expression host strain BL21(DE3) was transformed with pRSETB-ARU-Km^{r} as described (Sambrook et al. Molecular Cloning: A Laboratory Manual (1989)). One liter cultures were inoculated with 10 ml of overnight growth of Escherichia coli BL21(DE3) containing pRSETB-ARU-Km^{r} and grown at 37° C. in Terrific broth (Sigma, St. Louis, Mo.) containing 25 μg/ml of kanamycin to an O.D. 600 of 1.8-2.0 and isopropyl B-D-thiogalactopyranoside (IPTG) was added to a final concentration of 40 μM. Cells were harvested after 22 h of induction, suspended in 0.1 liter of standard phosphate buffered saline, pH 7.4, containing 0.2% casamino acids, and disrupted by sonication. Cellular debris was removed from the lysate by centrifugation. Lysates typically contained a titer (reciprocal of the highest dilution with an A₄₅₀ greater than 0.2) of 10⁶ in the TOX-A test EIA (TechLab, Inc., Blacksburg, Va.). Lysates were saturated with 40% ammonium sulfate, stirred at 4° C. overnight and precipitating proteins were harvested by centrifugation. The ammonium sulfate fraction was suspended in 0.1 liters of 5 mM K₂PO₄, 0.1 M NaCl₂, pH 8.0 and dialyzed extensively against the same buffer at 4° C. Insoluble material was removed by centrifugation. The dialyzed solution was passed through a column containing Sepharose CL-6B chromatography media (50 ml media/100 ml solution). Fractions were collected and monitored for the presence of rARU by EIA using the TOX-A test. Fractions containing EIA activity were analyzed by SDS-PAGE for the presence of rARU at a molecular weight of approximately 102 kDa. Fractions containing a single band of rARU were pooled. To further ensure purity the pooled solution was again passed over a Sepharose CL-6B column (25 ml media/100 ml protein solution). The solution containing purified rARU was filtered sterilized by passage through a 22μ filter and stored at 4° C. Purified rARU along with samples from the steps of purification (lysate and dialyzed ammonium sulfate fraction) are shown in FIG. 5. The procedure typically yields approximately 100 mg rARU per liter of E. coli/pRSETB-ARU-Km^{r} culture. A combined 6-liter batch yielded 0.850 liters of rARU at 0.88 mg/ml for a total of 748 mg of rARU or 125 mg/liter of culture. The amount of rARU recovered represented 23% of the total soluble protein.

### Example 3

Synthesis of Polysaccharide-rARU Conjugates.

Polysaccharides. Pneumococcal type 14 polysaccharide, Lot 40235-001, was manufactured by Lederle Laboratories, Pearl River, N.Y. S. flexneri type 2a O-specific polysaccharide and E. coli K1 polysaccharide were purified as described (Cohen, D. et al. Lancet 349:155-159 (1997); Devi et al. Proc. Natl. Acad. Sci. USA 88:7175-7179 (1991); Schneerson et al. Infect. Immun. 60:3528-3532 (1992)). All preparations had less than 1% protein and nucleic acid.

Chemicals. 1-ethyl-3-(3-dimethylaminopropyl) carboiimide, (EDC), succinic anhydride, MES (2-[N-morpholino]-thanesulfonic acid) hydrate, 2-[N-morpholino]-ethanesulfonic acid sodium salt), trinitrobenzenesulfonic acid (TNBS) and thimerosal, were from Sigma Co., St. Louis, Mo.; adipic acid dihydrazide, cyanogen bromide and acetonitrile, from Sigma-Aldrich, Milwaukee, Wis.; CL-4b and CL-6B Sepharose, Sephadex G-50, from Pharmacia, Piscataway, N.J.

Analytical methods. The protein and saccharide components of the conjugates were assayed as described (Chu et al. Infect. Immun. 59:4450-4458 (1991)). Derivatization with adipic acid dihydrazide was measured by the trinitobenzene sulfonic acid assay (Chu et al. Infect. Immun. 59:4450-4458 (1991)). The extent of succinylation was measured indirectly by the reduction in amino groups of rARU using lysine as a standard (Fields R. Biochem J. 124:581-590 (1971); Pavliokova et al. Infect. Immun. 67:5526-5529 (1999)).

Succinylation of rARU. Preliminary experiments defined the conditions that succinylated rARU while retaining its antigenicity as measured by double immunodiffusion with goat anti-CDTA (Pavliakova et al. Infect. Immun. 67:5526-5529 (1989)). Succinic anhydride was added to rARU at w/w of 1/10 at room temperature with mixing: the pH maintained at 7.2-7.5 with 0.5 M NaOH in a pH stat. After 20 minutes, the reaction mixture was passed through a 2.5×50 cm Sephadex G-50 column in 0.2 M NaCl and the void volume peak pooled and concentrated.

Conjugation of polysaccharides to rARU and rARUsucc. Pneumococcal type 14 polysaccharide and S. flexneri type 2a O-specific polysaccharide were activated with cyanogen bromide, derivatized with adipic acid dihydrazide, and bound to rARU or rARUsucc by water-soluble carboiimide condensation as described with the exception that the pH of the reactants was maintained with 0.1 MES, pH 6.0 (Chu et al. Infect. Immun. 59:4450-4458 (1991); Cohen, D. et al. Lancet 349:155-159 (1997); Schneerson et al. Infect. Immun. 60:3528-3532 (1992)). E. coli K1 polysaccharide was both derivatized with adipic acid dihydrazide and bound to rARU or rARUsucc by treatment with EDC (Devi et al. Proc. Natl. Acad. Sci. USA 88:7175-7179 (1991)). The composition of the adipic acid dihydrazide derivatized polysaccharides and of the conjugates is shown in Table 1. Note that low yields of conjugates, using rARU as the carrier, were obtained with the pneumococcal type 14 and S. fexneri type 2a polysaccharides. We were unable to synthesize a conjugate of the K1 polysaccharide with rARU.

TABLE 1
Composition of Clostridium difficile recombinant enterotoxin
A (rARU) conjugates of pneumococcal type 14 (Pn14), Escherichia
coli K1 (group B meningococcal) capsular polysaccharide and
Shigella flexneri type 2a O-specific polysaccharide.

| | Percent | | rARU/ | | |
|---|---|---|---|---|---|
| | adipic | rARU/ | polysaccharide | Yield (%) | |
| Conjugate | hydrazide | succinate | (w/w) | polysaccharide | rARU |
| Pn-14-rARU | 2.07 | NA | 0.52 | 10.4 | 5.2 |
| Pn-14 rARUsucc | 2.07 | 34.4 | 2.91 | 13.0 | 38.0 |
| SF-rARU | 5.50 | NA | 1.56 | 1.4 | 2.1 |
| SF-rARUsucc | 5.50 | 38.3 | 2.36 | 20.0 | 51.4 |
| K1-rARUsucc | 3.8 | 41.2 | 3.23 | 13.3 | 43.0 |

NA—Not available

### Example 4

Immune Response to Polysaccharide Component of the Conjugates.

Vaccination of mice. Female 5 weeks-old general purpose Swiss Albino mice at the NIH or outbred hsd/ICR mice (Harlan Sprague Derby, Inc., Indianapolis, Ind.) were injected subcutaneously with 0.1 ml containing 2.5 μg polysaccharide in the conjugate every 2 weeks. Mice (n=10) were exsanguinated 2 weeks after the first injection and 1 week after the second and third injections.

Serologic. IgG and IgM antibodies to S. flexneri type 2a LPS and to E. coli K1 polysaccharides were measured by ELISA as described (Chu et al. Infect. Immun. 59:4450-4458 (1991); Devi et al. Proc. Natl. Acad. Sci. USA 88:7175-7179 (1991)). IgG anti-pneumococcal type 14 polysaccharide were assayed by ELISA and total polysaccharide antibody by radioimmunoassay (RIA) and as described (Kayhty et al. J. Infect. Dis. 172:1273-1278 (1995); Schneerson et al. Infect. Immun. 60:3528-3532 (1992); Shiffman et al. J. Immunol. Methods 33:130-144 (1992)).

Pneumococcal type 14 (Pn14) antibodies (Table 2). Both conjugates (Pn14-rARU and Pn14-rARUsucc) elicited statistically significant rises of IgG antibodies after the first and the second injections (p<0.005). The third injection of both conjugates elicited rises in IgG (4.38 to 6.41 EU for Pn14-rARU and 6.10 to 9.76 EU for Pn14-rARUsucc) and IgM (4.82 to 7.57 for Pn14-rARU and 6.16 to 8.54 for Pn41-rARUsucc) but these were not statistically significant. Pneumococcal type 14 polysaccharide alone elicits only trace levels of antibodies in mice (Schneerson et al. Infect. Immun. 60:3528-3532 (1992)). PBS did not elicit Pn14 antibodies.

TABLE 2
Serum pneumococcal antibodies (Pn14) elicited in mice by conjugates composed
of Clostridium difficle recombinant toxin A repeating units (rARU) alone
or succinylated (rARUsucc) bound to pneumococcal type 14 polysaccharide.

| | ELISA (Geometric mean and 25-75 centiles) | | | | | |
|---|---|---|---|---|---|---|
| | IgG | | | IgM | | |
| | First | Second | Third | First | Second | Third |
| Conjugate | injection | injection | injection | injection | injection | injection |
| Pn14-rARU | 0.90^{a} | 4.38^{b} | 6.41^{c} | 2.32 | 4.82 | 7.57 |
| | 0.77-1.26 | 1.97-11.6 | 4.56-7.37 | 1.57-4.35 | 3.70-9.12 | 4.86-10.4 |
| Pn-14-rARUsucc | 0.71^{d} | 6.10^{e} | 9.76^{f} | 1.38 | 6.16 | 8.54 |
| | 0.42-1.65 | 3.55-7.40 | 7.10-12.4 | 0.59-2.0 | 4.37-9.41 | 6.41-9.66 |

c.b vs 0.90, f, e vs 0.71 p < 0.005;
c vs b, f vs e, NS;
f vs c, NS
6 wks-old mice were injected s.c. with 2.5 mg of pneumococcal type 14 polysaccharide as a conjugate at 2 wk intervals. Mice (n = 10) were exsanguinated 2 wks after the 1st injection and 7 days after the 2nd and 3rd injections and their sera assayed for IgG and IgM anti-pneumococcal type 14 polysacchride by ELISA. A hyperimmune serum, arbitrarily assigned a value of 100 ELISA units (EU) was the reference.

The correlation coefficients between the geometric mean levels of conjugate-induced pneumococcal type 14 polysaccharide antibodies for all post-vaccination sera, as measured by ELISA and RIA, were statistically significant (Table 3).

Shigella fexneri type 2 a (SF) IgG LPS antibodies (Table 4). Both SF-rARU

TABLE 3
Comparison between conjugate-induced serum
Streptococcus pneumoniae type 14 geometiric mean antibody
levels measured by RIA and ELISA (IgG).

| | Geometric mean | | | | |
|---|---|---|---|---|---|
| Conjugate | Injection | RIA | ELISA | r = | p = |
| Pn14-rARU | 1st | 723 | 0.90 | 0.73138 | 0.02 |
| Pn14-rARU | 2nd | 2232 | 4.38 | 0.97738 | 0.0001 |
| Pn14-rARU | 3rd | 3732 | 6.41 | 0.89505 | 0.0005 |
| Pn14-rARUsucc | 1st | 682 | 0.71 | 0.94647 | 0.0001 |
| Pn14-rARUsucc | 2nd | 3985 | 6.10 | 0.94233 | 0.0001 |
| Pn14-rARUsucc | 3rd | 5725 | 9.76 | 0.88912 | 0.0006 |

Pneumococcal type 14 antibodies were measured by ELISA expressed as units and by RIA expressed as ng antibody nitrogen/ml serum.

and SF-rARUsucc elicited LPS antibodies after the second injection compared to prevaccination levels (p=0.001). Reinjection for the third time elicited a rise of IgG anti-LPS for both conjugates but was statistically significant only for SF-rARUsucc (2.48 vs 0.37, p=0.04). The SF IgG anti-LPS levels induced by the two conjugates were not statistically different.

Escherichia coli K1 (meningococcus group B) IgG antibodies. K1-rARUsucc elicited a significant rise in antibodies after all 3 injections: first injection (1.35 EU), second (12.4 vs 1.35, p=0.0001) and third (104 vs 12.4, p=0.002).

TABLE 4
Serum LPS antibodies elicited in mice by Shigella flexneri 2a O-specific
polysaccharide (SF) bound to Clostridium difficile recombinant toxin
A repeating units (rARU) alone or succinylated (rARUsucc)

| | ELISA (Geometric mean and 25-75th centiles) | | | |
|---|---|---|---|---|
| | IgG | | IgM | |
| Immunogen | Second injection | Third injection | Second injection | Third injection |
| SF-rARU | 0.75 (0.40-1.43) | 1.61 (1.13-3.38) | 6.92 (4.85-12.2) | 7.18 (2.74-18.2) |
| SF-rARUsucc | 0.37 (0.03-1.63) | 2.48 (1.35-5.14) | 1.54 (0.18-54.5) | 4.06 (1.74-8.77) |

b vs a, p = 0.04
6 wks-old mice were injected subcutaneously with 2.5 mg of S. flexneri type 2a O-specific polysaccharide alone or as a conjugate at 2 wk intervals. Mice (n = 10) were exsanguinated 7 days after the second and third injections and their sera assayed for IgG anti-LPS by ELISA. A hyperimmune serum pool, arbitrarily assigned a value of 100 ELISA units (EU), served as a reference

### Example 5

Immune Response to the rARU Component of the Conjugates.

Antibodies to C. difficile toxin A (CDTA). Antibodies to native toxin A were measured by ELISA, with toxin A isolated from C. difficile as the coating antigen, and by in-vitro neutralization of cytotoxicity (Lyerly et al. Infect. Immun. 35:1147-1150 (1982)). Human intestinal epithelial HT-29 cells (ATCC HTB 38) were maintained in 96 well plates with McCoy's 5A medium supplemented with 10% fetal calf serum in a 5% CO₂ atmosphere. HT-29 cells were chosen because of their high sensitivity to CDTA probably because of the high density of the carbohydrate receptor on their surface. Serial 2-fold dilutions of sera were incubated with 0.4 μg/ml of CDTA for 30 min at room temperature. CDTA-serum mixtures were added to the wells at a final concentration of 20 ng of toxin A per well (about 200 times the minimal cytotoxic dose for HT-29 cells) in a final volume of 0.2 ml. The neutralization titer is expressed as the reciprocal of the highest dilution that completely neutralized cytotoxicity.

TABLE 5
Serum antibodies (mg/ml) to Clostridium difficile toxin A
(CDTA) elicited in mice by recombinant enterotoxin A (rARU) or
polysaccharides bound to rARU alone or succinylated (rARUsucc)

| | mg rARU | ELISA (Geometric mean and 25-75 centiles) | | |
|---|---|---|---|---|
| Conjugate | Injected | First injection | Second injection | Third injection |
| rARU* | 6.94 | ND | ND | 717 (621-863) |
| Pn14-rARU | 1.29 | 3.70 (2.55-5.08) | 80.1 (69.8-131) | 194 (113-236) |
| Pn14rARU succ | 7.30 | 7.94 (5.21-11.3) | 183 (146-175) | 371 (274-463) |
| SF-rARU | 3.90 | ND | 433 (258-609) | 613 (485-778) |
| SF-rARUsucc | 6.94 | ND | 191 (118-291) | 518 (366-615) |
| SF-rARU* | 3.90 | ND | ND | 437 (372-547) |
| SF-rARUsucc* | 6.94 | ND | ND | 242 (172-443) |
| K1 | 8.08 | 10.7 (6.75-17.2) | 84.9 (72.5-131) | 390 (279-470) |

183 vs 7.94 p = 0.0001,
371 vs 183 p = 0.0005,
80.1 vs 3.70 p = 0.0001,
194 vs 80.1 p = 0.007,
7.94 vs 3.70 p = 0.01,
183 vs 80.1 p = 0.004,
371 vs 194 p = 0.01
*hsd/ICR mice. Remainder were NIH SA mice.
ND (not done).
6 wks-old mice were injected SC with 2.5 mg of polysaccharide as a conjugate at 2 wk intervals. Groups of mice (n = 10) were exsanguinated 7 days after each injection and their sera assayed for anti-CDTA by ELISA.

All 5 conjugates elicited high levels of anti-CDTA (194-613 μg/ml) (Table 5). Since the 2.5 μg immunizing dose of the conjugates was based on its polysaccharide content, the amount of rARU injected was different for each conjugate. For example, on a protein weight basis, Pn14-rARU, with 1.29 μg of rARU, elicited 194 μg CDTA antibody/ml (150.3 μg Ab/μg rARU injected). In contrast, Pn14-rARUsucc, that contained 7.3 μg of rARU per dose, elicited 371 μg CDTA antibody/ml (50.8 μg Ab/μg rARUsucc injected). Pn14-rARU induced more anti-CDTA per μg rARU than Pn14-rARUsucc, however, the total amount of anti-CDTA elicited by Pn14-rARUsucc was greater due to its higher content of rARU. The difference between the levels of anti-CDTA elicited by Pn14-rARU (194 μg CDTA antibody/ml) compared with Pn14-rARUsucc (371 μg CDTA antibody/ml) was significant.

SF-rARU, containing 3.9 μg of rARU, elicited 437 μg CDTA antibody/ml (112.0 μg Ab/μg rARU injected) compared to 518 μg CDTA antibody/ml for SF-rARUsucc (34.9 μg Ab/μg rARUsucc injected). Although the specific immunogenic activity for the rARUsucc was lower than that of the rARU in the SF conjugates, there was no statistical difference between the levels of CDTA antibody elicited by the two conjugates (437 μg Ab/ml for SF-rARUsucc vs 242 μg Ab/ml for SF-rARU).

K1-rARUsucc, that elicited 390 μg CDTA antibody/ml, had comparable specific immunogenic activity of its rARU component (48 μg Ab/mi per μg rARUsucc).

### Example 6

CDTA Neutralizing Antibodies.

Individual sera obtained 7 days after the third injection of the conjugates were assayed individually for their neutralization of approximately 200 times the cytotoxic dose of CDTA on human intestinal epithelial HT-29 cells. All sera from the mice immunized with the conjugates had a neutralizing titer greater than or equal to 64. The geometric mean and range of neutralizing titers for each conjugate is shown in Table 6.

Conjugate-induced antibody levels approached or surpassed the neutralizing activity of an affinity-purified goat antibody, containing 0.5 mg/ml, that was raised against formalin inactivated CDTA.

TABLE 6
Serum neutralizing activity against the in vitro cytotoxicity
for HT-29 cells of Clostridium difficile toxin A (CDTA)

| | | |
|---|---|---|
| | | Reciprocol |
| | mg Ab/ml | neutralization titer |
| Immunogen | (ELISA) | (GM and range) |

| | | | |
|---|---|---|---|
| Pn14-rARU | 194 | 104 | 64-256 |
| Pn14-rARUsucc | 371 | 111 | 64-128 |
| SF-rARU | 613 | 194 | 64-256 |
| SF-rARUsucc | 518 | 181 | 64-256 |
| Goat antitoxin (0.5 mg/ml)* | | | 128 |
| PBS | | | 0 |

Neutralizing titers were the highest serum dilution that completely inhibited the cytotoxicity of CDTA (20 ng/well) on HT-29 cells. The titers represent the geometric mean of sera from general purpose Swiss Albino mice (n = 10) obtained 7 days after the 3rd injection. Anti-CDTA was measued by ELISA and the mean value expressed as mg Ab/ml serum.
*Affinity purified goat antibody

### Example 7

Protection Against Lethal Challenge with CDTA (Table 7).

Hsd/ICR mice were injected with SF-rARU, SF-rARUsucc or rARU as described in EXAMPLE 4 above. One week after the third injection, the mice were challenged intraperitoneally with a lethal dose (150 ng) of CDTA. Almost all mice vaccinated with either conjugate or rARU were protected. Based upon the amount of rARU injected, rARU and SF-rARU elicited similar levels of anti-CDTA. As expected, SF-rARUsucc elicited lower levels of anti-CDTA than the other two immunogens but the recipients were comparably protected.

This invention has been described by a direct description and by examples. As noted above, the examples are meant to be only examples and not to limit the invention in any meaningful way. Additionally, one having ordinary skill in the art to

TABLE 7
Protection of mice against lethal challenge with 150 ng
of Clostridium difficile toxin A (CDTA)^{a} inducued
by vaccination with polysaccharide-rARU conjugates

| | | | CDTA | Reciprocal |
|---|---|---|---|---|
| | mg rARU | Survivals/ | antibodies | neutralization |
| Immunogen | injected | total | (ELISA)^{b} | titer^{c} |
| rARU | 6.94 | 19/20 | 717 (621-863) | 128-256 |
| SF-rARU | 3.90 | 17/20 | 437 (372-547) | 128-256 |
| SF-rARUsucc | 6.94 | 19/20 | 242 (172-443) | 64-256 |
| PBS | 0 | 2/15 | Not determined | <2 |

^{a}Mice (hsd/ICR) injected I.P. with 150 ng of CDTA 7 days after the 3rd injection of rARU or conjugate.
^{b}Mean μg/ml antibody level (25-75 centiles) of sera used for pool (n = 10) from each group bled 4 h before challenge with CDTA.
^{c}Highest dilutions of sera (range) that completely neutralized the cytotoxicity of CDTA (20 ng/well) on HT-29 cells.

which this invention pertains in reviewing the specification and claims which follow would appreciate that there are equivalents to those claimed aspects of the invention. The inventors intend to encompass those equivalents within the reasonable scope of the claimed invention.

### LITERATURE CITED

U.S. Patent Documents

U.S. Pat. No. 4,863,852 (Wilkins et al.) (1989)
U.S. Pat. No. 5,098,826 (Wilkins et al.) (1992)
U.S. Pat. No. 4,530,833 (Wilkins et al.) (1985)
U.S. Pat. No. 4,533,630 (Wilkins et al.) (1985)
U.S. Pat. No. 4,879,218 (Wilkins et al.) (1989)
U.S. Pat. No. 5,736,139 (Kink et al.) (1998)
U.S. Pat. No. 5,919,463 (Thomas et al.) (1999)
   Other References
Lyerly, D. M. and T. D. Wilkins, in Infections of the Gastrointestinal Tract, Chapter 58, pages 867-891, (Raven Press, Ltd, New York 1995)
Moncrief et al., Infect. Immun. 65:1105-1108 (1997)
Barroso et al. Nucl. Acids Res. 18:4004 (1990)
Dove et al. Infect. Immun. 58:480-488 (1990)
Krivan et al., Infect. Immun. 53:573-581 (1986)
Tucker, K and T. D. Wilkins, Infect. Immun. 59:73-78 (1991)
Just et al. Nature 375:500-503 (1995)
Just et al. J. Biol. Chem 270:13932-13939 (1995)
Hofmann et al. J. Biol. Chem. 272:11074-11078 (1997)
Faust and Song. Biochem. Biophys. Res. Commun. 251:100-105 (1999)
Robbins, J. B. and R. Schneerson. J. Infect Dis. 161:821-832 (1990)
Robbins et al, JAMA 276:1181-1185 (1996)
Avery O T and Goebel W F J. Exp. Med. 50:533-550 (1929)
Goebel, W. F., J. Exp. Med. 69:353-364 (1939)
Buchanan-Davidson et al., J. Immunol. 83:543-555 (1959)
Fuchs et al., J. Biol. Chem. 240:3558-3567 (1965)
Chu et al. Infect. Immun. 59:4450-4458 (1991)
Devi et al., Infect. Immun. 59:732-736 (1991)
Devi et al., Infect. Immun. 59:3700-3707 (1990)
Fattom et al. Infect. Immun. 60:584-589 (1992)
Fattom et al. Infect. Immun. 61:1023-1032 (1993)
Kondadu et al. Infect. Immun. 62:5048-5054 (1994)
Kayhty et al. J. Infect. Dis. 172:1273-1278 (1995)
Szu et al. Infect. Immun. 54:448-453 (1986)
Szu et al. Infect. Immun. 59:4555-4561 (1991)
Szu et al., Infect. Immun. 57:3823-3827 (1989)
Anderson et al. J. Clin. Invest. 76:52059 (1985)
Cohen, D. et al. Lancet 349:155-159 (1997)
Dagan et al. Infect. Immun. 66:2093-2098 (1998)
Devi et al. Proc. Natl. Acad. Sci. USA 88:7175-7179 (1991)
Pavliakova et al. Infect. Immun. 67:5526-5529 (1999)
Claesson et al. J. Pediatr. 112:695-702 (1988)
Lagergard et al. Infect. Immun. 58:687-694 (1990)
Schneerson et al. Infect. Immun. 52:519-528 (1986)
Weinstein, R A. Emerg Infect. Dis. 4:416-420 (1998)
Kayaba et al. Surg Today 27:217-219 (1997)
Moreno et al. Clin Infect. Dis. 212:1308-1312 (1995)
Tenover et al. J. Hosp. Infect. 43 Suppl:S3-7 (1999)
Woodford et al. J. Antimicrob Chemother. 45:258-259 (2000)
Busch et al. J. Biol. Chem. 273:19566-19572 (1998)
Lindberg et al. Carbohydr. Res. 58:177-186 (1977)
Carlin et al. Eur. J. Biochem. 139:189-194 (1984)
Kenne et al. Eur. J. Biochem. 91:279-284 (1978)
Bhattacharjee et al. J. Biol. Chem. 250:1926-1932 (1975)
Sambrook et al. Molecular cloning: A Laboratory Manual (1989)
Phelps et al. Infect. Immun. 59:150-153 (1991)
Fields, R. Biochem. J. 124:581-590 (1971)
Schneerson et al. Infect. Immun. 60:3528-3532 (1992)
Chu et al. Infect. Immun. 59:4450-4458 (1991)
Shiffman et al. J. Immunol. Methods 33:130-144 (1992)
Lyerly et al. Infect. Immun. 35:1147-1150 (1982)

1. An immunogenic composition for eliciting an immune response to a pathogenic organism which composition comprises recombinant toxin B repeating units (rBRU) of Clostridium difficile and a polysaccharide component, wherein said polysaccharide component is an antigen of a pathogenic microorganism other than C. difficile, along with a pharmaceutically acceptable carrier.

2. The immunogenic composition of claim 1, wherein said polysaccharide component is a capsular polysaccharide or a lipopolysaccharide.

3. The immunogenic composition of claim 1, wherein said polysaccharide has been isolated from said pathogenic microorganism.

4. The immunogenic composition of claim 1, wherein said pathogenic microorganism is selected from the group consisting of: Streptococcus pneumoniae; Neisseria meningitidis; Escherichia coli; and Shigella.

5. The immunogenic composition of claim 4, wherein said pathogenic microorganism is Streptococcus pneumoniae.

6. The immunogenic composition of claim 4, wherein said pathogenic microorganism is Shigella.

7. The immunogenic composition of claim 4, wherein said pathogenic microorganism is Neisseria meningitidis.

8. The immunogenic composition of claim 4, wherein said pathogenic microorganism is Escherichia coli K1.

9. The immunogenic composition of claim 1, wherein said pathogenic microorganism is selected from the group consisting of: Staphylococcus aureus; coagulase-negative Staphylococcus; Enterococcus species; Enterobacter species; Candida species; and Pseudomonas species:

10. The immunogenic composition of claim 9, wherein said pathogenic microorganism is Staphylococcus aureus serogroup 5 or serogroup 8.

11. The immunogenic composition of claim 1, wherein said rBRU is conjugated to said polysaccharide.

12. A vaccine comprising the immunogenic composition of claim 1.

13. The vaccine of claim 12, wherein said vaccine is formulated for use in humans.

14. The vaccine of claim 12, wherein said vaccine is formulated for use in animals.

15. A vaccine comprising the immunogenic composition of claim 11.

16. The vaccine of claim 15, wherein said vaccine is formulated for use in humans.

17. The vaccine of claim 15, wherein said vaccine is formulated for use in animals.

18. A method to elicit an immune response in a subject to a pathogenic organism which method comprises injecting a subject in need of such response with an effective amount of the immunogenic composition of claim 1.

19. A method to elicit an immune response in a subject to a pathogenic organism which method comprises injecting a subject in need of such response with an effective amount of the immunogenic composition of claim 11.

20. A method to elicit an immune response in a subject to a pathogenic organism which method comprises injecting a subject in need of such response with an effective amount of the vaccine of claim 12.

21. A method to elicit an immune response in a subject to a pathogenic organism which method comprises injecting a subject in need of such response with an effective amount of the vaccine of claim 15.
